(19) United States
(12) Patent Application Publication
(10) Pub. No.: US 2026/0124073 A1
(43) Pub. Date: May 7, 2026

(54) Predictive Analytic Tool for Vision Quality Optimization

(71) Applicant: Tracey Technologies, Corp., Houston, TX (US)

(72) Inventor: Joe S. Wakil, Houston, TX (US)

(73) Assignee: Tracey Technologies, Corp., Houston, TX (US)

(21) Appl. No.: 19/377,291

(22) Filed: Nov. 3, 2025

Related U.S. Application Data
(63) Continuation-in-part of application No. PCT/US2024/027895, filed on May 4, 2024.
(60) Provisional application No. 63/500,255, filed on May 4, 2023.

(51) Int. Cl. A61F 9/008; A61B 34/10

(52) U.S. Cl. CPC A61F 9/00812; A61B 34/10; A61B 2034/104; A61B 2034/105; A61B 2034/108; A61F 2009/0087; A61F 2009/00878

(57) ABSTRACT

Provided herein is a predictive analytics tool used for the optimization of vision quality in a subject. The tool uses at least one of an optical quality index of a total eye, an optical quality index of a cornea or a dysfunctional lens index as preoperative conditions input into the tool. The output is a type and at least one outcome of a technology performed on an eye of the subject. A correlation between measured data of preoperative conditions from the cloud library and the input data of preoperative conditions for each component of pre-op conditions is calculated to enable recommendations by a doctor.

### CROSS-REFERENCE TO RELATED APPLICATION

This continuation-in-part application claims benefit of priority under 35 U.S.C. § § 120 and 365(c) of international patent application PCT/US 2024/027895, filed May 4, 2024, which claims benefit of priority under 35 U.S.C. § 119(e) of provisional patent application U.S. Ser. No. 63/500,255, filed May 4, 2023, the entirety of all of which are hereby incorporated.

### BACKGROUND OF THE INVENTION

### Field of the Invention

The present invention relates to the fields of ophthalmology and analytic tools useful in ophthalmologic endeavours. More particularly, the present invention is an ophthalmologic means used for eye examination and treatment, particularly, for eye surgery planning based on measurement of vision quality, as represented by its descriptive components: quality vision index (QVI), cornea performance index (CPI), and dysfunction lens index (DLI), prediction and optimization of surgery outcome, including the choice of the technique, its instrumental support, and objects to be replaced, for example, the intraocular lenses.

### Description of the Related Art

Replacement of the cataractous crystalline lens with the intraocular implant at the end of 40-s by H. Ridley stimulated the search for adequate description of the quality of human vision. F. W. Cambell and R. W. Gubisch in their pioneering studies of the optical quality of the human eye. (J. Physiol., 1966, 186, pp. 558-578) used the modulation transfer function and its Fourier transform, that is linespread function.

J. Liang and D. R. Williams paid attention to the role of higher-order aberrations on the retinal image quality (J. Opt. Soc. Am. A, 1997, 11, pp. 2873-2883). In the U.S. Pat. No. 7,357,509, D. R. Wlliams, et al. proposed several metrics to predict the subjective impact of the eye's wavefront aberrations based on wavefront errors or slopes, the area of the critical pupil, a curvature parameter, the point spread function, the optical transfer function, or the like. Other techniques include the fitting of a sphero-cylindrical surface, the use of multivariate metrics, and customization of the metric for patient characteristics such as age.

These metrics are insufficient for making prediction for the success of the planned surgery. Parameters of the replacing intraocular lenses and the technologies used for implantation are not taken into account. The experience of similar cases is also to be included into the planning schedule.

K. Angelides proposed a management system for developing individualized health improvement plans that includes the internet usage for an individual participant facing a chronic disease or consistent discomfort (U.S. Pat. No. 10,199,126). Its principle is directed to storing the current information on the status of the patient and getting continuously the outside advising.

In case of planning an ophthalmologic surgery, doctor needs to use stored experience and data purposely acquired. Adequate for clinical usage is empirical indexing of the quality of vision based on quality vision index QVI, cornea performance index CPI, and dysfunction lens index DLI, calculated from measured aberrations that take into account empirically confirmed inter-aberration interaction of higher-order aberrations of the cornea and of the crystalline lens (F. Faria-Correia, et al. J Refract Surg. 2016, 32, pp. 244-248).

Thus, there is a recognized need in the art for an improved means for determining visual corrections and planning a surgery based on the same. Particularly, the prior art is deficient in a predictive analytics tool that utilizes descriptive components of a quality vision index (QVI), a cornea performance index (CPI), and a dysfunction lens index (DLI). The present invention fulfills this long-standing need and desire in the art.

### SUMMARY OF THE INVENTION

The present invention is a predictive analytic tool for vision surgery planning that includes a big data multiple input—multiple output (MIMO) cloud library, an aberrometer with a wave front analyzer, a central processing unit, a multiplexer, a wi-fi modem, and a demultiplexer. On the pre-op stage, based on the aberrometer collected data, the wave front analyzer provides the central processing unit with an index of the optical quality of the eye, an index of the optical quality of the cornea, an index of the lens optical dysfunction, and data on the tear film conditions. The central processing unit, upon getting the requested access to the MIMO cloud library, allows the wi-fi modem to transfer the data from the wave front analyzer to the multiplexer and to upload the data to the cloud library through one of its open multiple inputs. Through one of the open multiple-access cloud library outputs, the central processing unit gets the access to the data stored in the cloud library and chooses the pre-op data maximally correlated with the current patient's data. The requested data are downloaded to the central processing unit through the wi-fi modem and the demultiplexer. Operating interactively through the central processing unit, doctor adjusts the patient's conditions and the goals of the planned surgery to the post-op results, achieved with the surgery techniques, materials and instruments used by the surgeons of a certified level. After the surgery has been made, the post-op results accompanied by the surgery requisites are uploaded to the cloud library.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the above-recited features of the invention are to be understood in detail, more particular descriptions of the invention briefly summarized above are illustrated in the appended drawings. These drawings form a part of the specification. However, that the appended drawings illustrate preferred embodiments of the invention, they are not to be considered limiting in their scope.

FIG. 1 is a schematic of the electronic components comprising the predictive analytics tool.

FIG. 2 illustrates a reduced visual function via the Dysfunction Lens Index (DLI).

FIGS. 3A-3D illustrate stages I (FIG. 3A), II (FIG. 3B), III (FIG. 3C), a spectrum of states I, II and III (FIG. 3D) of a cataract via the Dysfunction Lens Index.

FIG. 4 illustrates the angle alpha and angle kappa distances in the eye.

FIGS. 5A-5B illustrate optical alignment measurements in a subject's eye with a low angle alpha (FIG. 5A) and an extremely high angle alpha (FIG. 5B).

FIGS. 6A-6D illustrate an example of a corneal performance result (FIG. 6A), a corneal axial map (FIG. 6B), corneal performance at various radii (FIG. 6C), and corneal performance scores (FIG. 6D).

FIG. 7 illustrates an example of a total optical performance result.

FIGS. 8A-8H illustrate lens pathologies in three subjects left (OS) eyes (FIGS. 8A-8B, 8E-8F and right (OD) eyes (FIGS. 8C-8D, 8G-8H) pre-operative (FIGS. 8A, 8C, 8E, 8G) and postoperative (FIGS. 8B, 8D, 8F, 8H) surgery.

FIGS. 9A-9C illustrate corneal pathologies in three subjects.

FIGS. 10A-10C illustrate postoperative problems.

FIGS. 11A-11B show population statistics for CPI performance with an absolute count (FIG. 11A) and relative frequency (FIG. 11B).

FIGS. 12A-12C show population statistics for Quality of Vision (QVI)performance with an absolute count (FIG. 12A), relative frequency (FIG. 12B) and the distribution of QVI scores for a population for each of the 4^{th} to 10^{th} age decile (FIG. 12C).

FIGS. 13A-13D show the distribution of scores for each of postoperative, preoperative and no surgery populations for the Corneal Performance Index (CPI) (FIG. 13A), the Dysfunction Lens Index (DLI) (FIGS. 13B-13C) and Quality of Vision Index (QVI) (FIG. 13D).

FIGS. 14A-14C are Placido images illustrating initial rings (FIG. 14A), loss of sharpness (FIG. 14B) and discontinuity (FIG. 14C).

FIGS. 15A-15C illustrate Tear Film Analysis of a normal tear film (FIG. 15A) and a dry eye (FIG. 15B) and a Tear Film Analysis display (FIG. 15C).

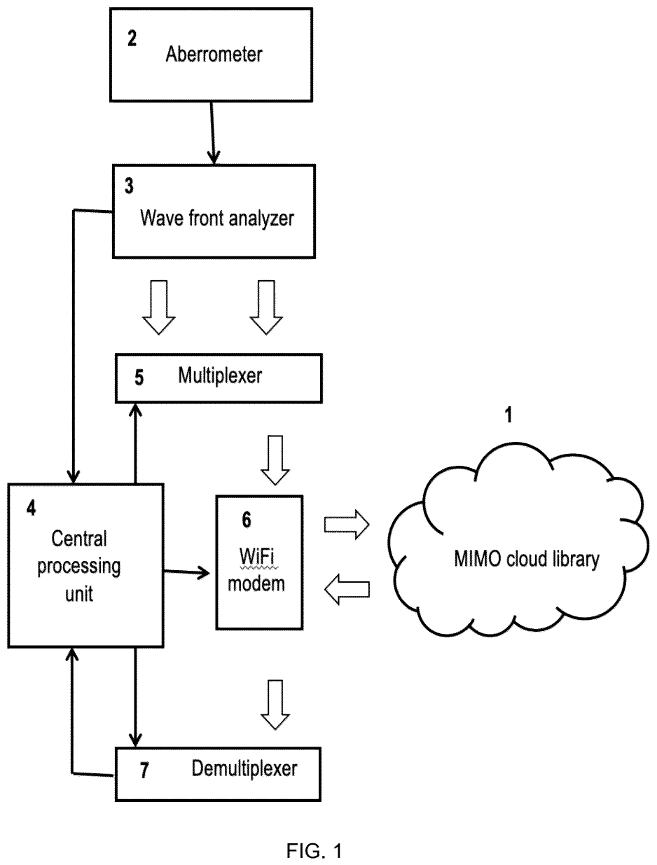

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, “patient” and “subject” are used interchangeably.

In one embodiment of the invention there is provided a predictive analytics tool for vision quality optimization in a subject, comprising a big data multiple input—multiple output (MIMO) cloud library with password access for authorized users thereof, said multiple input data containing at least one component of preoperative conditions and said multiple output data containing a type and at least one outcome of a technology performed on an eye of the subject.

In this embodiment, the at least one component may comprise an optical quality index of a total eye, an optical quality index of a cornea or a dysfunctional lens index. Also in this embodiment the multiple input data of preoperative conditions may be measured for the subject to be treated. In addition, a correlation may be calculated between measured data of preoperative conditions and the multiple input data of preoperative conditions from the cloud library for each component of pre-op conditions. Furthermore at least one case with a highest correlation of preoperative conditions may be selected from the cloud library, and a ranking thereof may be defined in a multi-dimensional vector space for each type and the at least one outcome for the technology performed within the multiple output data from the cloud library. Further still a list of recommended technologies may be output for a final approval by a doctor.

The invention provided herein utilizes higher order aberration data in the form of an index based on proprietary measures of the cornea's optical plane defined at the cornea's anterior surface including its tear film where the internal optics higher order aberrations is determined by the subtraction of the cornea's to the entire eyes higher order aberrations. Accumulated scores of each of these three indices, the CPI, DLI and QVI in preoperative and postoperative eyes as well as eyes with pre and post corrective means as with contact lenses or spectacles etc. can provide an table of data for future comparison and analysis of patients results. These indices are quantified in a 0 -10 numeric value for such comparisons.

Examples of patients visual results at certain points in time either before or after any procedure either surgical or not may provide such tabular raw data in creating a data set given a patients age, sex and other demographics including patients'refractive errors. The quality of vision as denoted by the lack of higher order aberrations or its reduction can now be measured for each component of cornea, internal optics, and total eye at the retinal plane with a difference of the value between pre-and post-procedure. The preoperative and postoperative exams accumulated over thousands if not millions of cases form the raw data to provide a predictive tool in ascertaining the most likely outcome for new patients undergoing similar procedures or receiving specific implants such as, but not limited to, intraocular lenses.

The predictive analytic tool for vision surgery planning consists of a big data multiple input—multiple output (MIMO) cloud library 1, an aberrometer 2, a wave front analyzer 3, a central processing unit 4, a multiplexer 5, a wi-fi modem 6, and a demultiplexer 7. The MMIMO cloud library 1 has multiple inputs for receiving the information and data from qualified clients and multiple outputs for sending them the information stored in its big database. The pre-op data of the current patient are acquired by the aberrometer 2 and properly processed by its wave front analyzer 3, both usually being constructively in a common housing, such as the iTrace instrument of Tracey Technologies, Corp., TX. The wave front analyzer 3 is connected electrically with the central processing unit 4, to which it delivers the pre-op data of the current patient and from which it gets commands to start communication with the multiplexer 5. Wi-fi modem 6 is controlled by the central processing unit 4 getting commands on uploading the patient's pre-op data from the wavefront analyzer 3 and other accompanying information, including patient's personal information from central processing unit 4 to the cloud library 1. Alternatively the predictive analytic tool is functional without the multiplexer and demultiplexer.

On the pre-op stage, based on the aberrometer collected data, the wave front analyzer provides the central processing unit with an index of the optical quality of the eye, an index of the optical quality of the cornea, an index of the lens optical dysfunction, and data on the tear film conditions. The central processing unit, upon getting the requested access to the MIMO cloud library, allows the wi-fi modem to transfer the data from the wave front analyzer to the multiplexer and to upload the data to the cloud library through one of its open multiple inputs. Through one of the open multiple-access cloud library outputs, the central processing unit gets the access to the data stored in the cloud library and chooses the pre-op data maximally correlated with the current patient's data. The requested data are downloaded to the central processing unit through the wi-fi modem and the demultiplexer. Operating interactively through the central processing unit, doctor adjusts the patient's conditions and the goals of the planned surgery to the post-op results, achieved with the surgery techniques, materials and instruments used by the surgeons of a certified level. After the surgery has been made, the post-op results accompanied by the surgery requisites are uploaded to the cloud library.

Other and further aspects, features, benefits, and advantages of the present invention will be apparent from the following description of the presently preferred embodiments of the invention given for the purpose of disclosure.

### EXAMPLE 1

### Dysfunctional Lens Index (DLI)

The Dysfunctional Lens Index (DLI) is a singular number that is calculated from the internal optics of the eye with the vast majority contributor being the lens (natural human crystalline lens or a man-made intraocular lens). The DLI is calculated from higher order aberrations within the internal optics of the eye as calculated by an aberrometer, most preferably through a true forward Ray Tracing aberrometer device, but can similarly be generated through other similar wavefront type devices such as those using a Hartmann-Shack sensor. The DLI scores the quality of vision from the Internal Optics (being predominately the lens) on a 0 to 10 scale. A lower number correlates to a more dysfunctional lens that negatively effects the quality of vision for the patient, which can indicate an early cataract. Essentially a low DLI correlates with an increase in the magnitude of higher order aberrations and predominantly is associated with early cataract formation (especially when below 5.0—see reference) in those patients over 50 years of age. Evaluating the DLI after cataract surgery is also a useful tool in evaluating the quality of vision and performance of man-made intraocular lenses as if DLI is high and close to 10 then that indicates very good optical lens performance with a DLI score of q0 being ideal promoting excellent vision. It is important to note that the DLI along with all the Quality of Vision indices do not count on the measurement of refractive error from sphere and cylinder. FIG. 2 shows a DLI scale where green on the color scale is “good” and warmer colors equate to reduced visual function. The letter “E” image reflects quality of vision from internal optics (Lens).

### Dysfunctional Lens Syndrome

Stage I of the aging lens occurs from the mid 40s to early 50s with a loss of accommodation and lens aberrations. In this stage the lens hardens and loses accommodation, i.e., presbyopia, lens high order aberrations develop with DLI<5 and quality of vision decreases. Stage II of the aging lens occurs from the mid 50s to the 60s and includes a forward lens scatter of light, in addition to stage I symptoms. In this stage there are increased lens higher order aberrations where DLI<4, small Lens protein aggregation causes forward light scatter, i.e., glare, and the patient sees bright colors as more “Brown”. Stage III of the aging lens occurs from the 60s to the 80s and includes a backscatter of light and lens opacity (cataract) in addition to stages I and II symptoms. In this stage there is a large lens protein aggregation which causes a backscatter of Light or opacity and a vision function decline with poor contrast sensitivity, and glasses are unable to correct to any normal acuity.

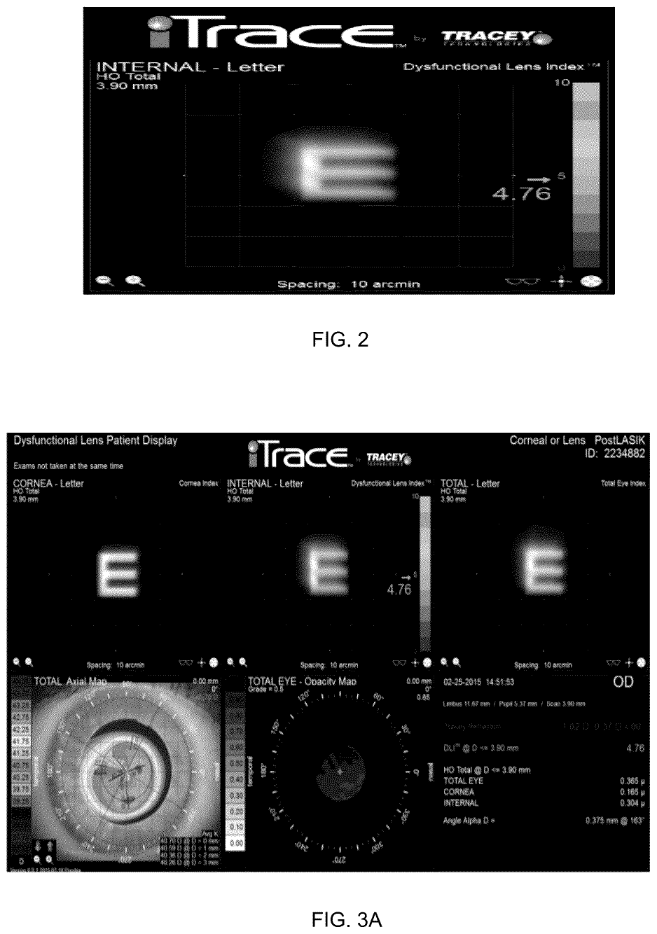

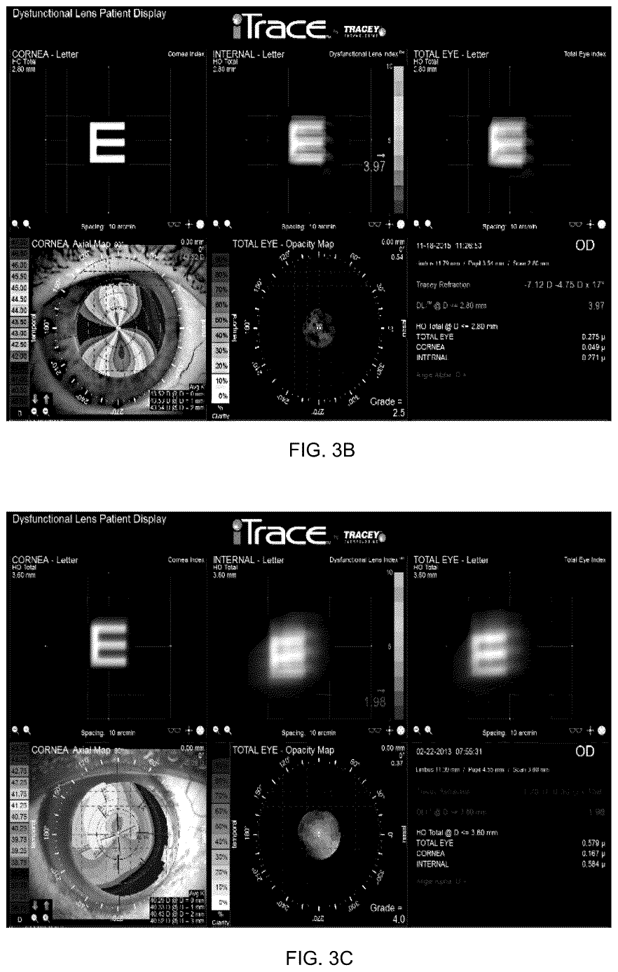

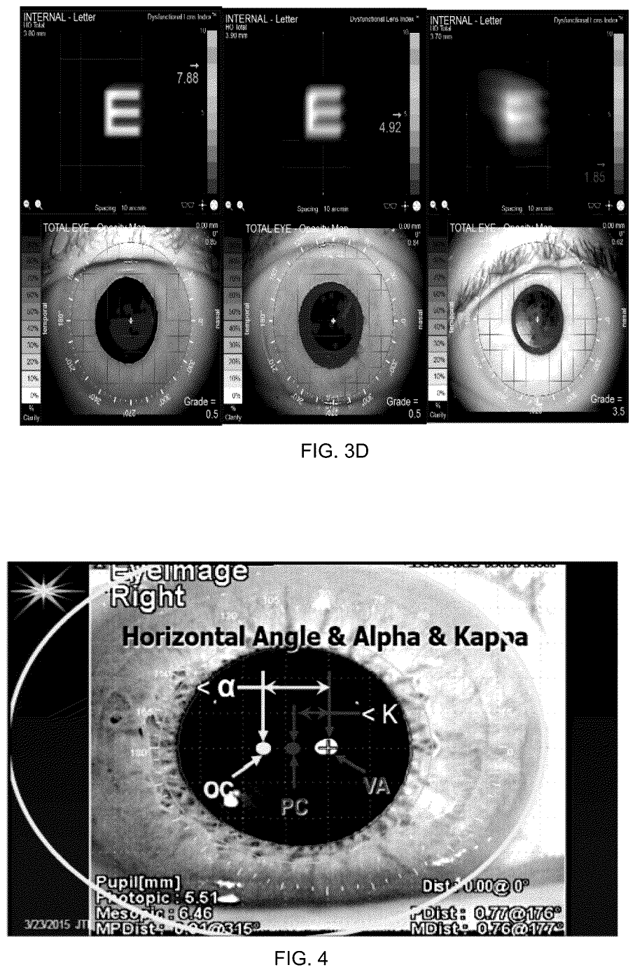

FIGS. 3A-3D show representative DLI patient displays. FIG. 3A shows a stage I early cataract in a 45 year old patient. FIG. 3B shows a stage II cataract with observable opacity and low DLI score. The cornea will support a toric Premium Lens. FIG. 3C shows a stage III cataract with an extremely low DLI score and high opacity grade (white area). The cornea is questionable with irregularities for optical support of a Premium lens. FIG. 3D is a DLI spectrum of aging lens stages I, II, and III.

### Optical Alignment Measurement

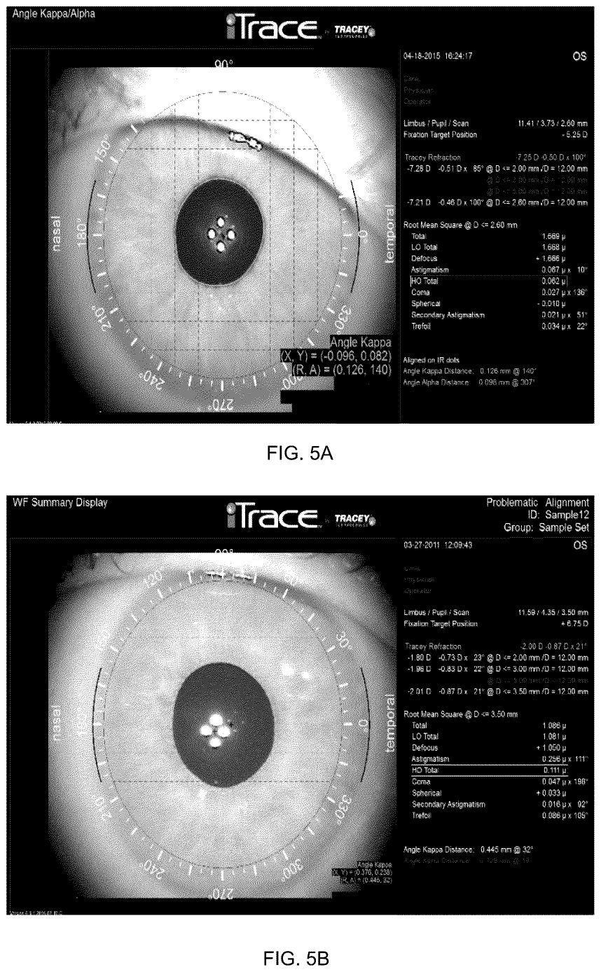

FIG. 4 shows the locations of the angle alpha (α) and angle kappa (κ) as the distances from the vertical axis (VA) to the optical center (OC) of the cornea and to the center of the pupil (PC), respectively. FIGS. 5A-5B are two cases that illustrate good optical alignment with low angle alpha in a first subject and an extremely high angle alpha in a second. Respectively, these subjects are a good candidate for multifocal intraocular lens (MIOL) and a poor candidate for MIOL.

### EXAMPLE 2

### Corneal Performance Index (CPI)

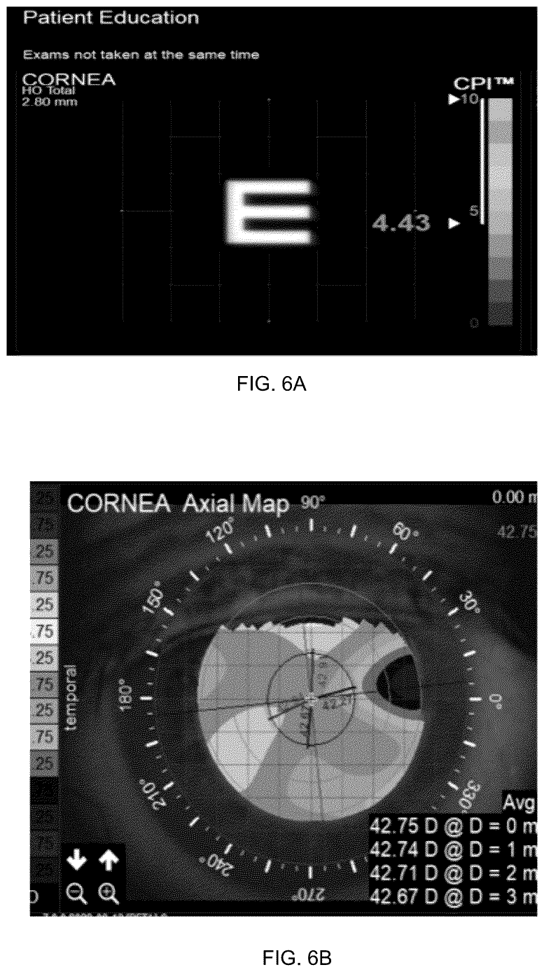

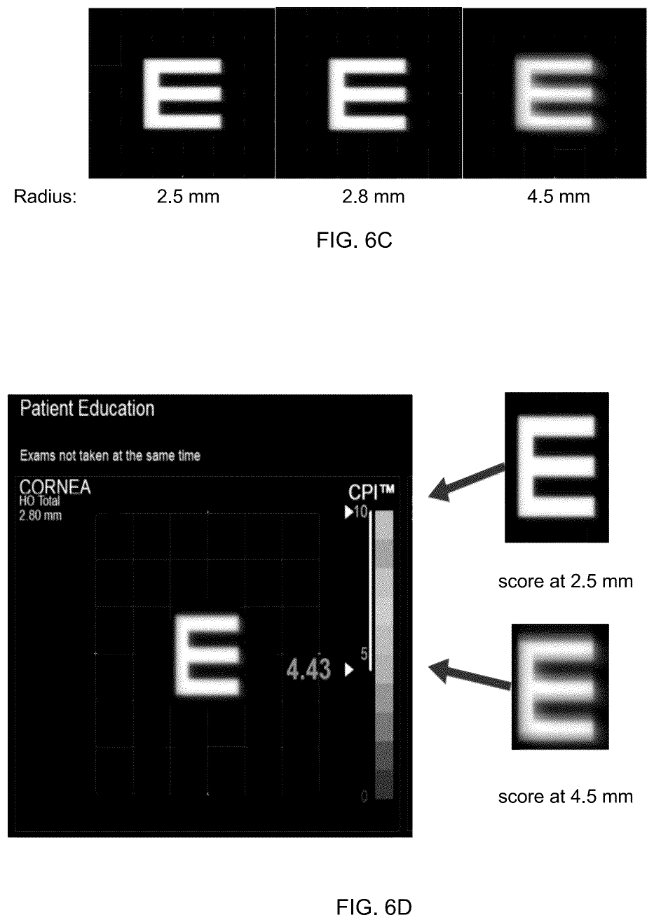

The Corneal Performance Index (CPI™) is a singular number that scores the quality of vision effects of the higher order aberrations generated at the cornea's anterior surface (FIG. 6A), which is a critical contribution to the patient's overall vision. The cornea's optical power (including the tear film on the anterior surface of the cornea) generates the majority of the refractive (focusing) power of the eye. This quality of vision index for the cornea is on a 0 to 10 scale with a bar that displays how that score would adjust based on the pupil size; for example, from 2.50 mm to 4.00 mm, or for any chosen pupil size typically up to 6.0 mm in diameter. The CPI number is automatically calculated from the cornea's higher order aberrations and is typically for the larger pupil sizes (4-6 mm) representing nighttime vision as this generally is the worst-case scenario with the lowest CPI as 10 is ideal for perfect vision. However, the CPI, if so chosen, can also be affected by the optical alignment between the cornea pupil and natural crystallin lens as this can be critical for determining premium IOL candidacy and selecting IOL types, such as multifocal, toric, aspheres or monofocals. The CPI creates a new standard in assisting eye care practitioners to determine if custom procedures are needed such as LASIK or special contact lenses to provide the patient with the best possible Quality of Vision after normal spectacle correction of spherical and cylindrical refractive error.

Corneal performance index grades the optical quality of the cornea by analyzing high order aberrations that degrades optical quality, it factors in optical alignment and pupil size as well for day and night analysis (FIG. 6A). The CPI is a Quality of Vision index by looking at different radii and get the corneal performance pertaining to that radius (FIG. 6B). The CPI takes the corneal topography, crops it at different radii between 2.5 to 4.5 mm, then calculates a score for each one of these points (FIG. 6C). The CPI attempts to address this by looking at different radii and get the corneal performance pertaining to that radius (FIG. 6D).

### EXAMPLE 3

### Quality of Vision Index

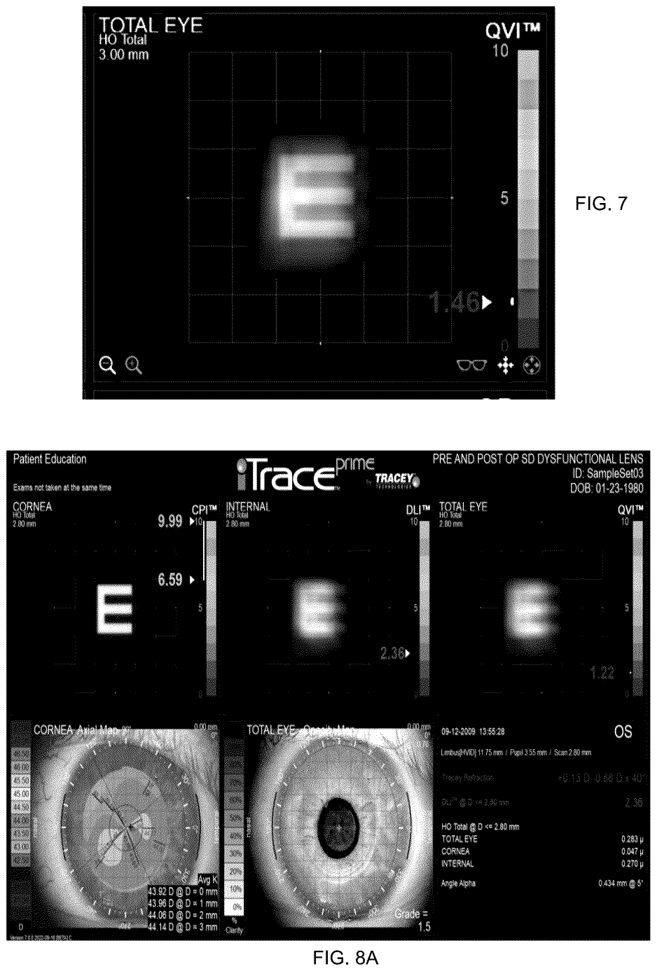

The Quality of Vision Index (QVI) is a number from 0 to 10 that scores the quality of vision for the total eye utilizing the higher order aberrations that are measured by ray tracing or wavefront technologies at the retinal plane. So this QVI does not involve the upper level processing of the visual cortex in the vision process as it is strictly based on the optical properties of the eye in generating an image at the retinal plane. As such the QVI utilizes only high order aberration information to generate its score. This Quality of Vision index is designed to correlate with patient's vision satisfaction after wearing standard optical correction; such as standard spectacles or eyeglasses correcting low order refractive errors, which are spherical errors in the common case of myopia and hyperopia and with or without astigmatism that would require cylinder correction. The QVI is a 0 to 10 score for visual performance to identify with patient complaints that today are not met through the typical 20/20 Snellen Visual Acuity standard. Many patients are 20/20 but unhappy with their vision as they still have some blur or halos, etc. The QVI, or any other namesake such as a WVI, is designed to objectively quantify such visual function disturbances patients experience every day or night with and without spectacle correction providing for a new Quality of Vision standard of care. The QVI quantifies the total optical performance of the total eye as it pertains to the optical image focused onto the retinal plane, primarily the macula, and may be selected for specific pupil sizes or for an overall value (FIG. 7).

### EXAMPLE 4

### Lens and Corneal Pathologies and Postoperative Problems

### Examples of Lens Pathologies in 3 Subjects

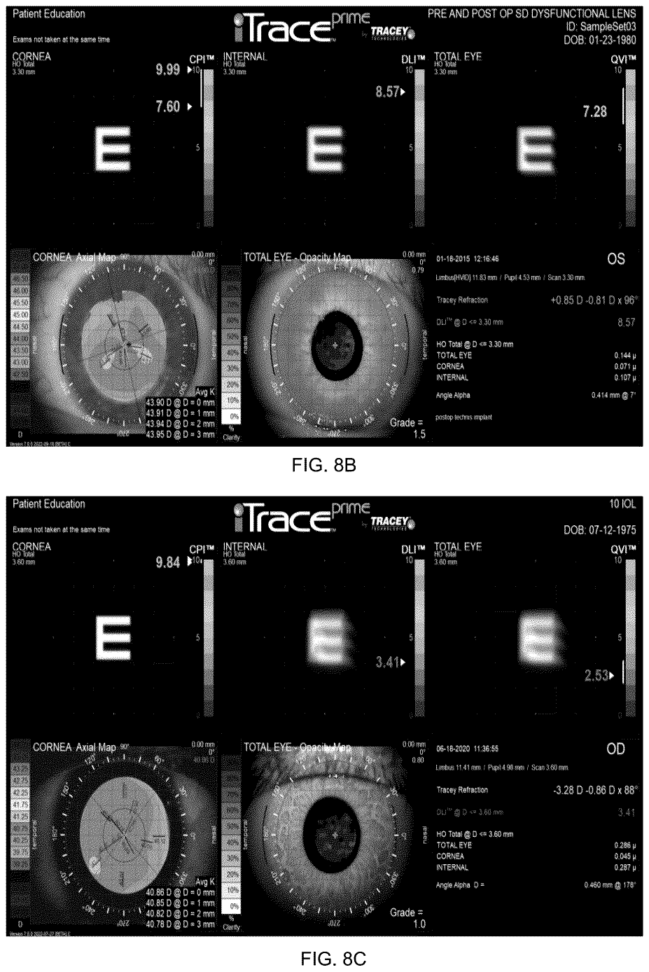

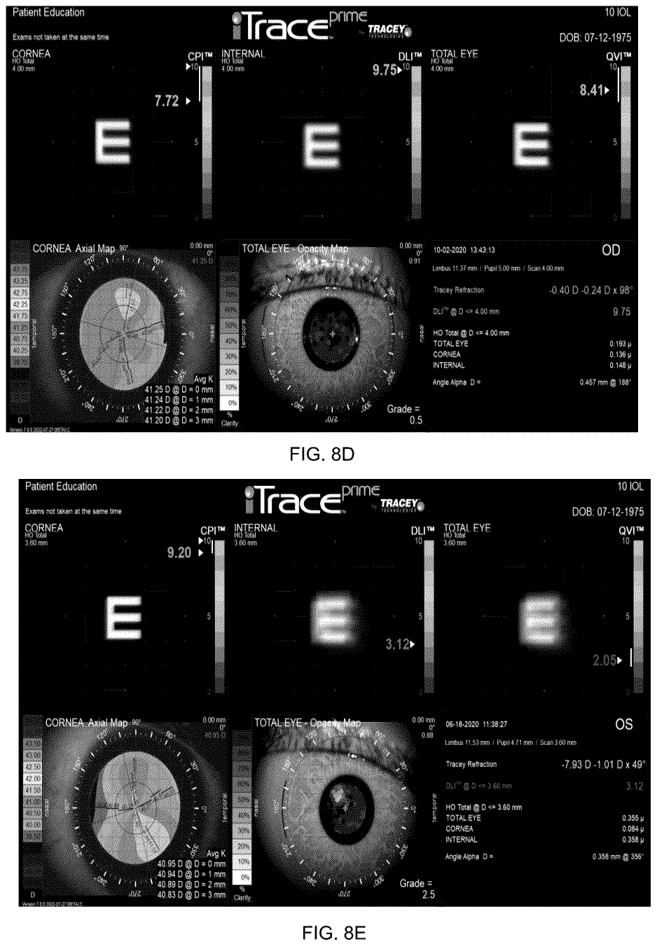

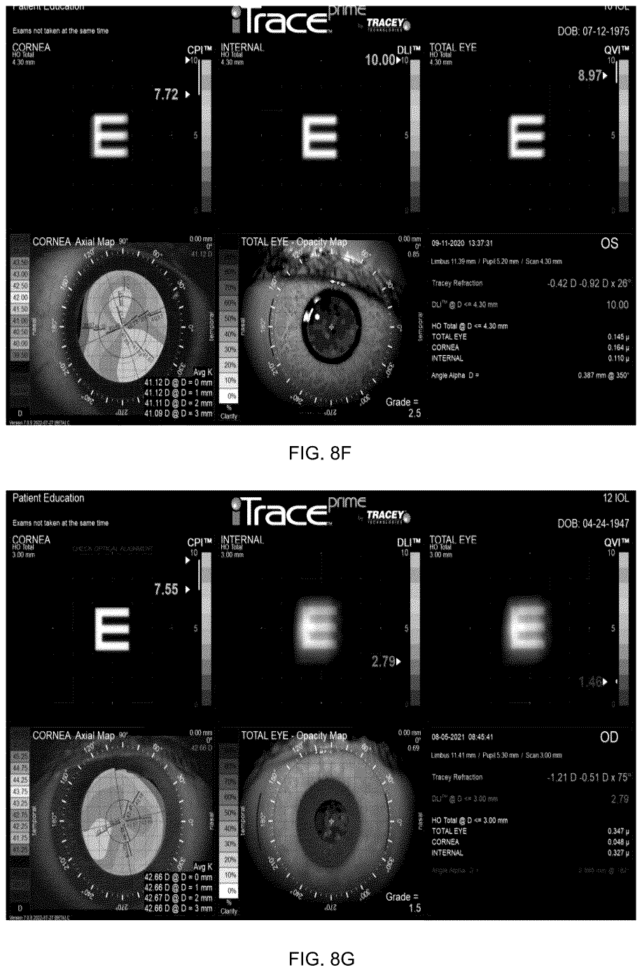

In case 1 the left eye (OS) is an example of a dysfunctional lens (FIG. 8A). There are no opacities on a slit lamp examination, but the patient cannot see well. FIG. 8B shows the postoperative improvement in the same patient.

In case 2 the right eye (OD) shows a preoperative early onset cataract (FIG. 8C) and the cataract postoperative with a Symfony ZXR00 intraocular lens (FIG. 8D) in the patient. The left eye early onset cataract in the patient is shown preoperatively in FIG. 8E and postoperatively with a Tecnis Multifocal ZLB00 in FIG. 8F.

In case 3 the preoperative patient has a cataract and treated dry eye (Keratoconjunctivitis sicca) (FIG. 8G) and postoperatively has a Symfony ZXR00.

### Examples of Corneal Pathologies in 3 subjects

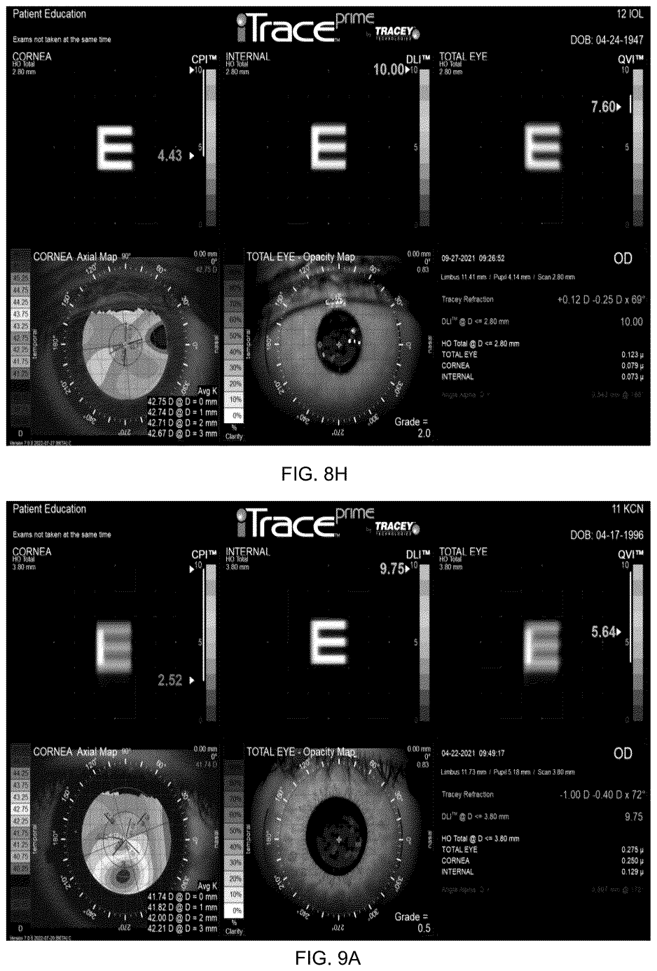

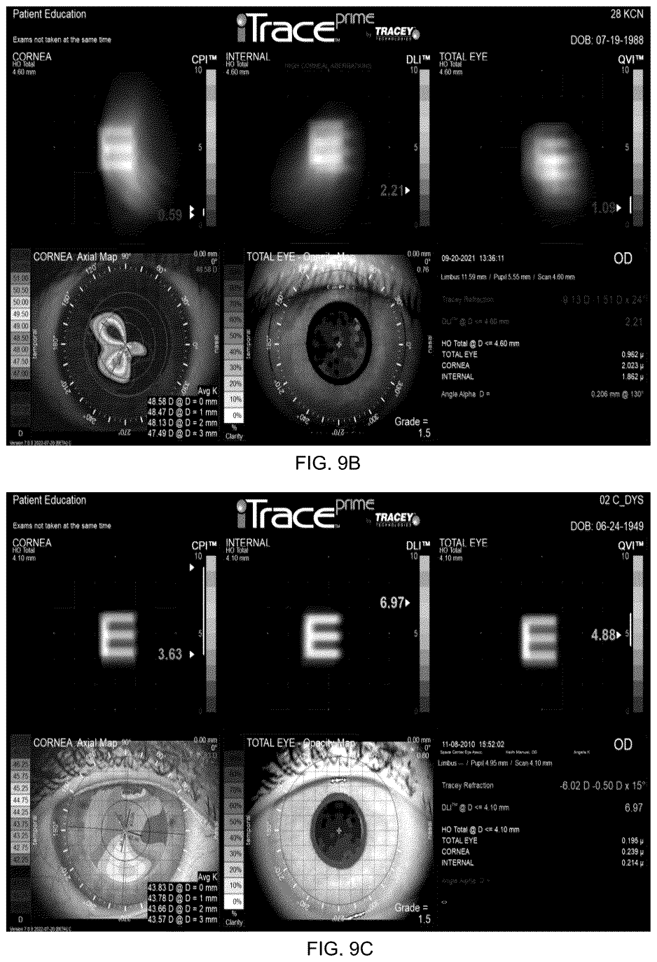

In case 4 a patient is keratoconic with moderate corneal and total vision performance (FIG. 9A).

In case 5 a patient is keratoconic with poor corneal and total vision performance (FIG. 9B).

In case 6 a patient has Reiss Buckler corneal dystrophy (FIG. 9C).

### Troubleshooting Post Operative Problems

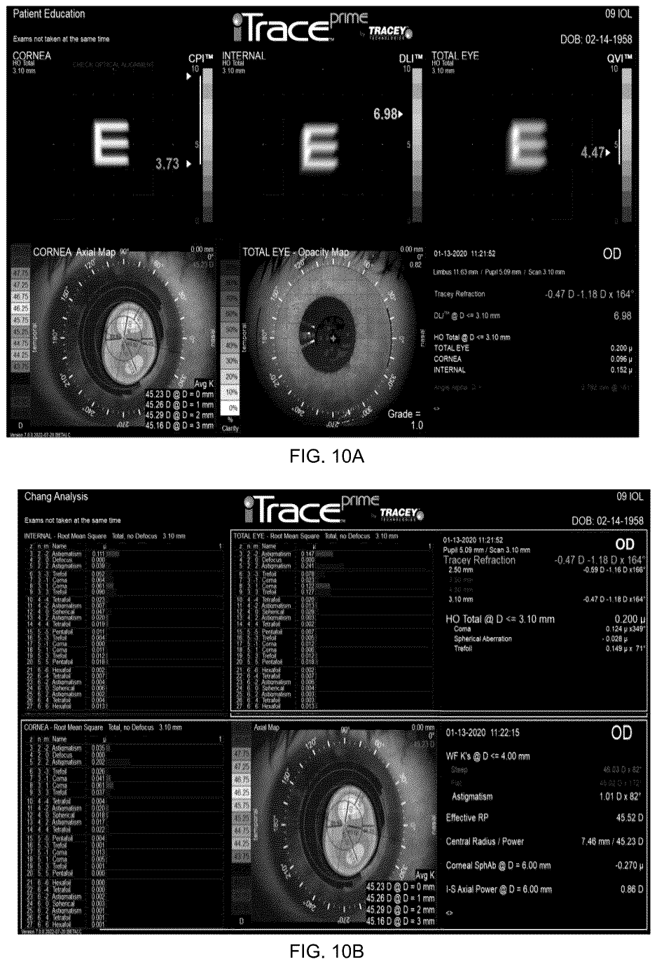

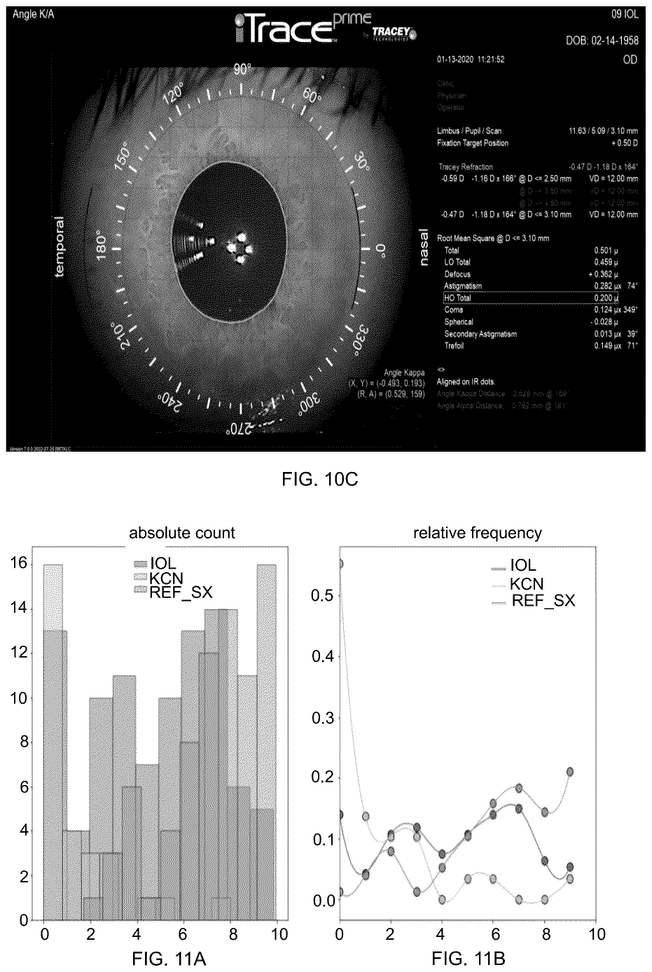

In case 7 a patient with hyperopic ablation undergoes refractive lens exchange with a Tecnis Premium ZLB00 IOL. Postoperatively the total eye performance is not great with little variation between day and night. The problem is that the aberrations in the cornea and lens are adding to each other (FIG. 10A). A closer look at the aberrations profile shows that the lens is adding to the problem of the total vision of this patient. The two main issues with the total vision are astigmatism and coma (FIG. 10B). The patient's optical alignment at least in part explains the coma and internal cylinder which is seen in this patient given that the patient had a non toric IOL (FIG. 10C).

CPI, DLI and QVI provide quality of vision solutions. They quantify the “intangible” aberrations of the eye that impact and reduce QOV, demonstrates when lens optics “compensates or decompensates” the cornea optics or when both are additive, and correlates to patient vision satisfaction and provides for predictive analytics to select best vision correction treatment and when to customize to optimize QOV.

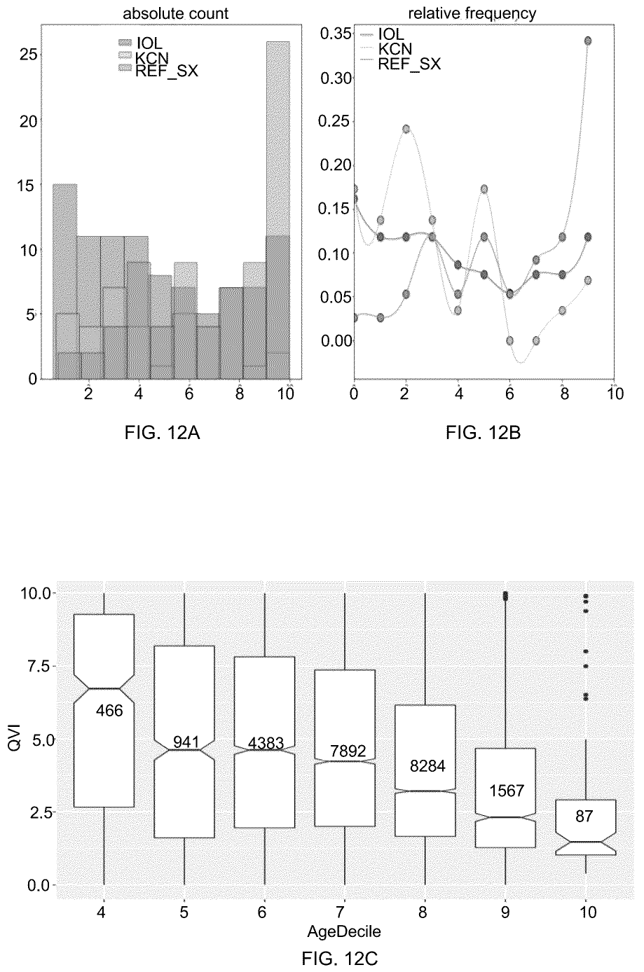

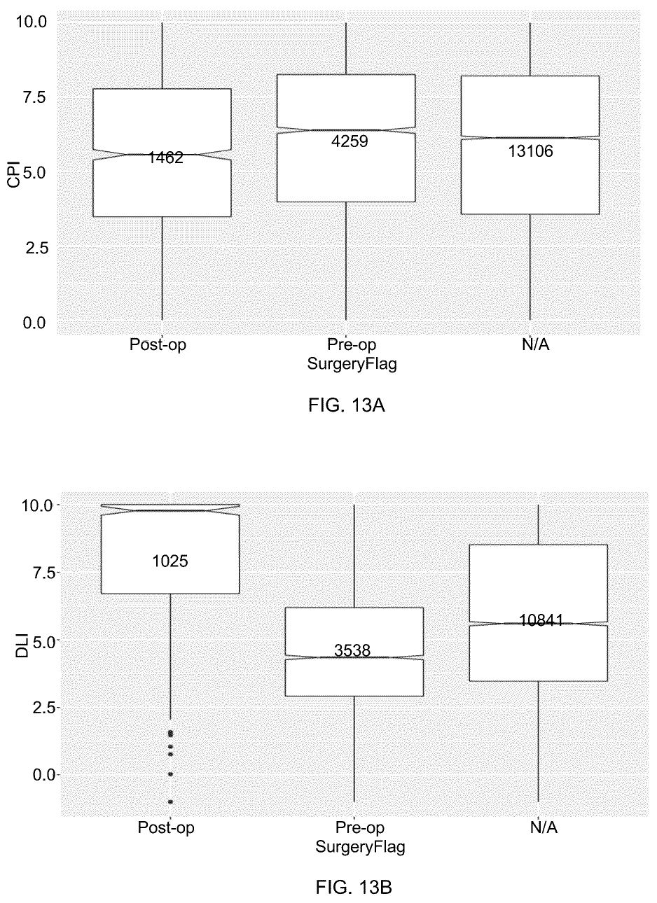

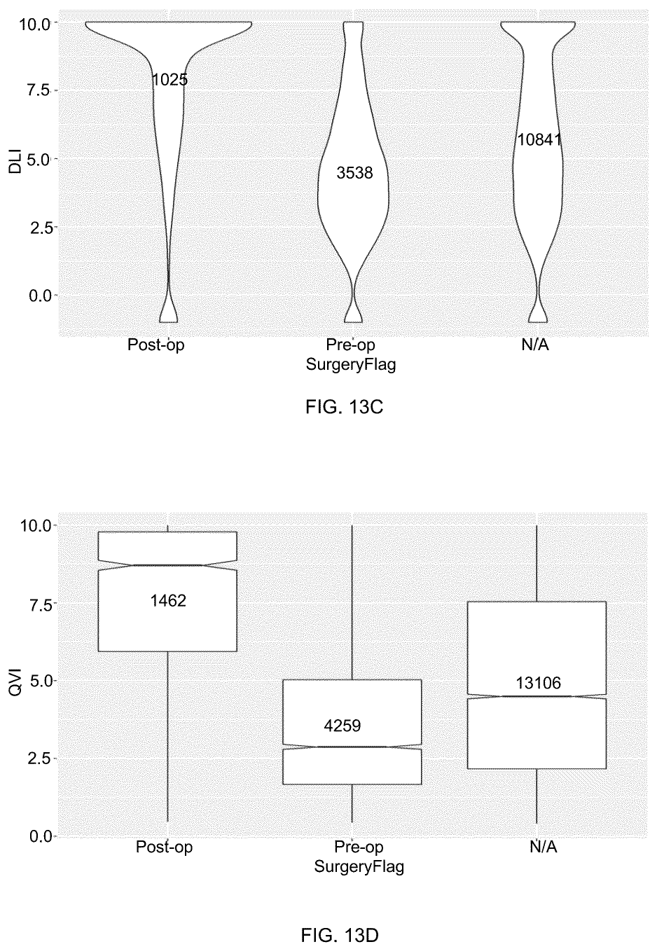

How these indices perform in different populations was determined. The performance of CPI in different populations is shown in FIGS. 11A-11B and that of QVI in FIGS. 12A-12B including illustrating the distribution of the QVI scores according to age deciles (FIG. 12C). In addition the scores for each of the CPI (FIG. 13A), DLI (FIGS. 13B-13C) and QVI (FIG. 13D) indices are distributed among postoperative, preoperative and no surgery populations.

### EXAMPLE 5

### Tear Function Index (TFI)

The Tear Function Index is a comprehensive index, with a single number score provided between 0 and 10, quantifying tear film quality and stability as it relates to visual function. A score of 10 is ideal as to provide excellent vision quality since the tear is a provider of optical surface performance of the cornea.

Besides tear film quality, TFI considers:
1. Spatial distribution of tear film quality (Central tear film changes within 4 mm are located within the pupil is more important for vision) and a two-dimensional map utilizing colors, as desired, can be generated over the cornea's surface for visualization.
2. Tear film dynamics: Stability over time or time to peak stability after a blink can incorporated into the TFI score

The TFI utilizes analysis of the Placido image off of the cornea surface but can also include other measures of optical or physical properties of the eye including assessment of tear film volume and/or in evaluating levels of aqueous or lipid content.

New tear film analysis software is used with the Tear Film Index (TFI) to help diagnose ocular surface disease and to analyze the vision quality effect. The goal is to differentiate between corneal irregular astigmatism and ocular surface disease and to guide the doctor and patient to the best treatment protocol.

### Tear Film Analysis With Placido Images

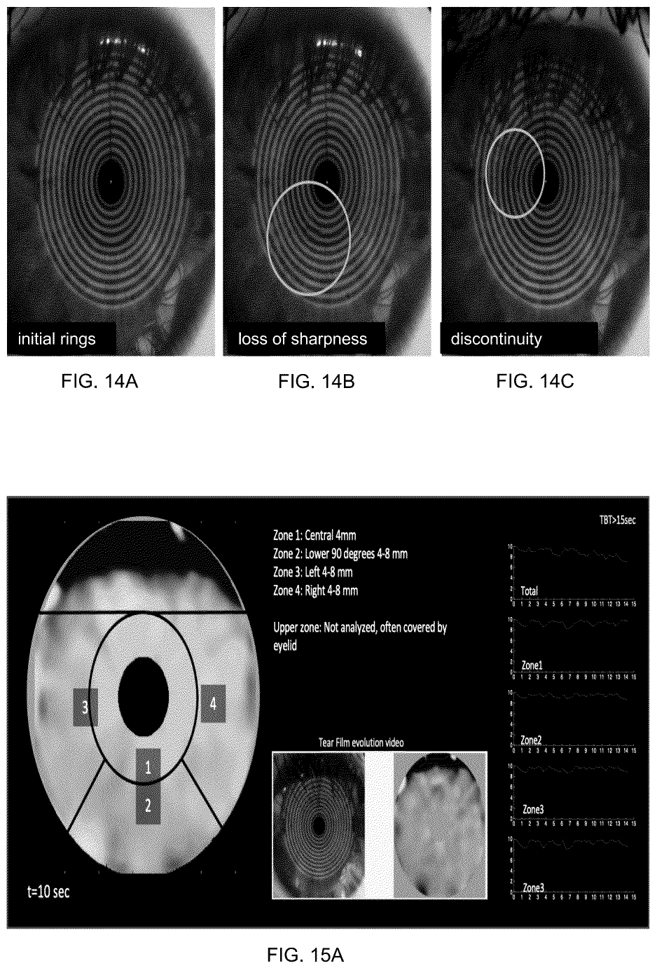

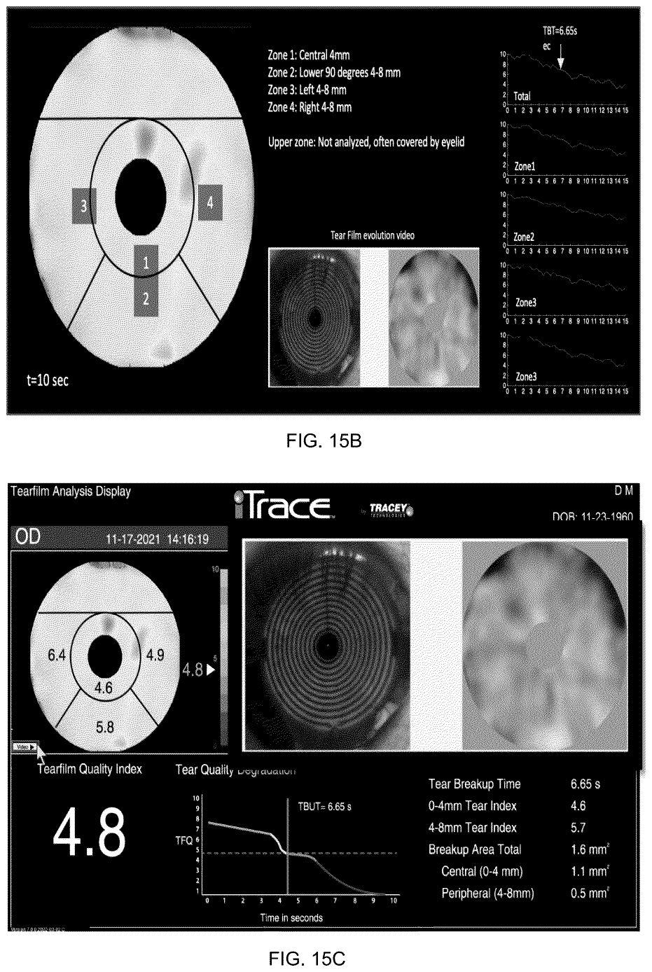

FIG. 14A shows a Placido ring. Analysis of a sequence of Placido images detects ring sharpness (FIG. 14B) and discontinuity (FIG. 14C). The TFI with Placido images provides an objective measurement of ring quality over time using Fourier domain analysis, an objective topographic tear breakup time and an automatic focus/alignment verification and blink rejection. FIG. 15A shows a normal tear film and FIG. 15B shows a film of a dry eye. A tear film analysis display is shown in FIG. 15C.

What is claimed is:

1. A predictive analytics tool for vision quality optimization in a subject, comprising:
a big data multiple input—multiple output (MIMO) cloud library with password access for authorized users thereof, said multiple input data containing at least one component of preoperative conditions and said multiple output data containing a type and at least one outcome of a technology performed on an eye of the subject.

2. The predictive analytics tool of claim 1, wherein the at least one component comprises an optical quality index of a total eye, an optical quality index of a cornea or a dysfunctional lens index.

3. The predictive analytics tool of claim 1, wherein said multiple input data of preoperative conditions are measured for the subject to be treated.

4. The predictive analytics tool of claim 1, wherein a correlation is calculated between measured data of preoperative conditions and the multiple input data of preoperative conditions from the cloud library for each component of pre-op conditions.

5. The predictive analytics tool of claim 1, wherein at least one case with a highest correlation of preoperative conditions is selected from the cloud library, and a ranking thereof is defined in a multi-dimensional vector space for each type and the at least one outcome for the technology performed within the multiple output data from the cloud library.

6. The predictive analytics tool of claim 5, wherein a list of recommended technologies is output for a final approval by a doctor.
